(19) United States
(12) Patent Application Publication
(10) Pub. No.: US 2010/0168249 A1
(43) Pub. Date: Jul. 1, 2010

(54) COMPOSITION CONTAINING REDUCED COENZYME Q10, AND METHOD FOR STABILIZING THE COMPOSITION

(75) Inventors: Shiro KITAMURA, Hyogo (JP); Takahiro UEDA, Osaka (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KANEKA CORPORATION, Osaka (JP)

(21) Appl. No.: 12/710,888

(22) Filed: Feb. 23, 2010

Related U.S. Application Data
(63) Continuation-in-part of application No. PCT/JP2008/065048, filed on Aug. 22, 2008.

(30) Foreign Application Priority Data
Aug. 23, 2007 (JP) 2007-216896

(51) Int. Cl. A61K 31/122; A61K 8/35; A23D 9/007

(52) U.S. Cl. 514/690; 426/606; 426/106; 426/125

(57) ABSTRACT

The invention provides a composition containing reduced coenzyme Q₁₀, which is useful as a pharmaceutical product, food, cosmetic, animal drug, feed, and the like, as well as a stabilization method thereof. The oxidation of reduced coenzyme Q₁₀ into oxidized coenzyme Q₁₀ by molecular oxygen can be suppressed by setting, in a composition containing reduced coenzyme Q₁₀ and at least one kind of oil component selected from the group consisting of fat, oil, a mixture thereof, fatty acid, wax and a surfactant, the weight ratio of the reduced coenzyme Q₁₀ to the oil component to not less than 1/4 when the composition is a liquid, and not less than 1/9 when the composition is a solid.

### TECHNICAL FIELD

The present invention relates to a composition containing reduced coenzyme Q₁₀ and a stabilizing method thereof. The reduced coenzyme Q₁₀ is a compound useful as food, food with nutrient function claims, food for specified health uses, health food, supplement, beverage, nutritional supplement, nutritional product, animal drug, feed, pet food, cosmetic, pharmaceutical product, therapeutic drug, prophylactic drug and the like.

### BACKGROUND ART

It is known that reduced coenzyme Q₁₀ can be obtained, for example, by a method comprising producing coenzyme Q₁₀ by a conventionally known method such as synthesis, fermentation, extraction from a naturally occurring substance and the like, and concentrating a reduced coenzyme Q₁₀ fraction in an eluate from chromatography and the like (see patent document 1). In this case, the patent document describes that oxidized coenzyme Q₁₀ contained in the above-mentioned reduced coenzyme Q₁₀ can be reduced with a general reducing agent, and concentrated by chromatography, and that the reduced coenzyme Q₁₀ can also be obtained by a method comprising reacting highly pure coenzyme Q₁₀ containing the existing oxidized type as a main ingredient with the reducing agent. However, the reduced coenzyme Q₁₀ obtained as described above cannot be always obtained at high purity, and tends to be obtained, for example, as a crystal, an oily product or a semi-solid material of low purity containing impurities including oxidized coenzyme Q₁₀. Thus, the present inventors have established and disclosed a production method for obtaining reduced coenzyme Q₁₀ of high quality (e.g., patent documents 2-4).

However, reduced coenzyme Q₁₀ is easily oxidized by molecular oxygen into oxidized coenzyme Q₁₀, and even in a case where reduced coenzyme Q₁₀ of high quality is produced by a method as described in the above-mentioned patent document, stabilization of reduced coenzyme Q₁₀ has been remained as an important issue when it is processed into a food, food with nutrient function claims, food for specified health use, nutritional supplement, nutritional product, animal drug, beverage, feed, cosmetic, pharmaceutical product, therapeutic drug, prophylactic drug and the like, or a material or composition therefor, and/or preserved after processing. Complete removal or blocking of oxygen during the above-mentioned processing and preservation is extremely difficult, and remaining or admixed oxygen particularly during heating for processing and long-term preservation exerts a markedly adverse effect. Such oxidation is directly related to quality problems such as the by-product oxidized coenzyme Q₁₀.

Therefore, stabilization of easily-oxidizable reduced coenzyme Q₁₀, namely, protection from oxidation, is an extremely important problem. Conventionally, addition of a reducing agent is a general method known for maintaining the stability of reduced coenzyme Q₁₀. However, the reducing agents to be used for the method include some agents inappropriate for food and pharmaceutical products, and many of them afforded only insufficient stability. For example, patent document 5 disclosing a composition concurrently containing a reducing agent and a production method thereof provides no detailed description relating to the quality of reduced coenzyme Q₁₀ contained in the composition and effect on the stability thereof and the like, and the achievable level of stabilization is not clear. In addition, the above-mentioned composition and a preparation method thereof are extremely complicated or troublesome. Moreover, the above-mentioned composition and a preparation method thereof are not entirely safe because the reaction mixture is used as it is. In addition, ascorbic acids to be used as reducing agents are oxidized to produce a considerable amount of dehydroascorbic acids, and the dehydroascorbic acids get mixed in with the above-mentioned composition such as foods, posing a problem. Dehydroascorbic acids and oxalic acid produced by decomposition from dehydroascorbic acids are highly noxious, unlike ascorbic acids. For example, an increased amount of lipid peroxide and a decreased amount of antioxidants in the liver and kidney, and an increased amount of oxalic acid in the kidney have been reported, and side effects such as decreased resistance to oxidation stress, easy onset of ureteral lithiasis (non-patent document 1) and the like are feared.

The present inventors considered that the above-mentioned problems have been solved, and have disclosed a composition which does not substantially inhibit stabilization of reduced coenzyme Q₁₀ and contains reduced coenzyme Q₁₀, a fat and oil (excluding olive oil) and/or a polyol, and if necessary, a polyglycerol ester of fatty acid (patent document 6). Under conditions requesting further oxidation stability, such as long-term preservation, preservation under harsh environment and the like, however, oxidation stability was not necessarily sufficient by this method alone.

patent document 1: JP-A-10-109933 (U.S. Pat. No. 6,184,255)
patent document 2: WO03/06408 (U.S. Pat. No. 7,145,044)
patent document 3: WO03/06409 (US2004/0214301)
patent document 4: WO03/32967 (US2005/0008630)
patent document 5: WO01/52822
patent document 6: WO03/062182 (US2005/0147598)
non-patent document 1: Nutrition Research, vol. 13, pp. 667-676, 1993

### DISCLOSURE OF THE INVENTION

### Problems to be Solved by the Invention

In view of the above, the present invention aims to provide a composition capable of suppressing oxidation of reduced coenzyme Q₁₀ and maintaining the stability, and a stabilizing method thereof.

### Means of Solving the Problems

The present inventors have conducted intensive studies in an attempt to solve the aforementioned problems and found that, in a composition comprising a mixture of reduced coenzyme Q₁₀ and oil components such as fat, oil, a mixture thereof, fatty acid, wax and a surfactant, oxidation of the reduced coenzyme Q₁₀ by molecular oxygen is preferably suppressed both when the composition is in a liquid state and a solid state by setting the weight ratio of the reduced coenzyme Q₁₀ to the oil components to a value not less than a certain value, which resulted in the completion of the present invention.

Accordingly, the present invention relates to the following [1]-[42].

[1] A liquid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/4 .

[2] The composition of the above-mentioned [1], wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower oil, kapok oil, evening primrose oil, shea butter, sal butter, cacao butter, sesame oil, safflower oil, olive oil, lard, milk fat, fish oil, beef fat, medium-chain triglyceride, and fat, oil and a mixture of fat and oil obtained by processing them.

[3] The composition of the above-mentioned [1], wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

[4] The composition of the above-mentioned [1], wherein the wax is at least one kind selected from the group consisting of beeswax, rice bran wax, carnauba wax, candelilla wax, sugar cane wax, shellac wax and jojoba wax.

[5] The composition of the above-mentioned [1], wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid and polyglycerol ester of condensed ricinoleic acid.

[6] The composition of any one of the above-mentioned [1] to [5], which is filled in a container.

[7] The composition of the above-mentioned [6], wherein the container comprises plastic, metal or glass as a material thereof.

[8] The composition of the above-mentioned [6] or [7], wherein the weight thereof per one package unit is not less than 1 kg.

[9] A method of stabilizing a liquid composition comprising reduced coenzyme Q₁₀ and at least one kind of oil component selected from the group consisting of fat, oil, a mixture thereof, fatty acid, wax and a surfactant, comprising setting a weight ratio of the reduced coenzyme Q₁₀ to the oil component to not less than 1/4.

[10] The method of the above-mentioned [9], wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower oil, kapok oil, evening primrose oil, rhea butter, sal butter, cacao butter, sesame oil, safflower oil, olive oil, lard, milk fat, fish oil, beef fat, medium-chain triglyceride, and fat, oil and a mixture of fat and oil obtained by processing them.

[11] The method of the above-mentioned [9], wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

[12] The method of the above-mentioned [9], wherein the wax is at least one kind selected from the group consisting of beeswax, rice bran wax, carnauba wax and candelilla wax.

[13] The method of the above-mentioned [9], wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid and polyglycerol ester of condensed ricinoleic acid.

[14] The method of any one of the above-mentioned [9] to [13], further comprising filling the composition in a container.

[15] The method of the above-mentioned [14], wherein the container comprises plastic, metal or glass as a material thereof.

[16] The method of the above-mentioned [14] or [15], wherein the composition is filled in the container under an oxygen-blocked atmosphere and is tightly sealed.

[17] A solid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/9.

[18] The composition of the above-mentioned [17], wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower oil, kapok oil, evening primrose oil, shea butter, sal butter, cacao butter, sesame oil, safflower oil, olive oil, lard, milk fat, fish oil, beef fat, medium-chain triglyceride, and fat, oil and a mixture of fat and oil obtained by processing them.

[19] The composition of the above-mentioned [17], wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

[20] The composition of the above-mentioned [17], wherein the wax is at least one kind selected from the group consisting of beeswax, rice bran wax, carnauba wax, candelilla wax, sugar cane wax, shellac wax and jojoba wax.

[21] The composition of the above-mentioned [17], wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid and polyglycerol ester of condensed ricinoleic acid.

[22] The composition of any one of the above-mentioned [17] to

[21], having a volume of not less than 0.02 cm³.

[23] The composition of any one of the above-mentioned [17] to

[22], which is packed with a film.

[24] The composition of any one of the above-mentioned [17] to

[23], which is filled in a container.

[25] The composition of the above-mentioned [24], wherein the container comprises plastic, metal or glass as a material thereof.

[26] The composition of any one of the above-mentioned [23] to

[25], wherein the weight thereof per one package unit is not less than 1 kg.

[27] A method of stabilizing a solid composition comprising reduced coenzyme Q₁₀ and at least one kind of oil component selected from the group consisting of fat, oil, a mixture thereof, fatty acid, wax and a surfactant, comprising setting a weight ratio of the reduced coenzyme Q₁₀ to the oil component to not less than 1/9.

[28] The method of the above-mentioned [27], wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower kerel oil, kapok oil, evening primrose oil, shea butter, sal butter, cacao butter, sesame oil, safflower oil, olive oil, lard, milk fat, fish oil, beef fat, medium-chain triglyceride, and fat, oil and a mixture of fat and oil obtained by processing them.

[29] The method of the above-mentioned [27], wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

[30] The method of the above-mentioned [27], wherein the wax is at least one kind selected from the group consisting of beeswax, rice bran wax, carnauba wax, candelilla wax, sugar cane wax, shellac wax and jojoba wax.

[31] The method of the above-mentioned [27], wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty so acid ester, polyglycerol ester of fatty acid and polyglycerol ester of condensed ricinoleic acid.

[32] The method of any one of the above-mentioned [27] to [31], further comprising setting the volume of the composition to not less than 0.02 cm³.

[33] The method of any one of the above-mentioned [27] to [32], further comprising packing the composition with a film.

[34] The method of any one of the above-mentioned [27] to [33], further comprising filling the composition in a container. [35] The method of the above-mentioned [34], wherein the container comprises plastic, metal or glass as a material thereof.

[36] The method of the above-mentioned [34] or [35], wherein the composition is filled in the container under an oxygen-blocked atmosphere and is tightly sealed.

[37] The method of the above-mentioned [33], wherein the film is an oxygen permeation suppressive film.

[38] A composition comprising reduced coenzyme Q₁₀ and fatty acid and/or diglyceride, wherein the reduced coenzyme Q₁₀ is obtained by reducing oxidized coenzyme Q₁₀ in the composition.

[39] The composition of the above-mentioned [38], wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

[40] The composition of the above-mentioned [38], wherein the diglyceride is at least one kind selected from the group consisting of monoglycerol dicaprylate, monoglycerol dicaprate, monoglycerol dilaurate, monoglycerol dimyristate, monoglycerol dipalmitate, monoglycerol distearate, monoglycerol dioleate, monoglycerol dilinoleate, monoglycerol dilinolenate, monoglycerol dibehenate and monoglycerol dierucate.

[41] The composition of the above-mentioned [38], wherein the weight ratio of the total amount of reduced coenzyme Q₁₀ and oxidized coenzyme Q₁₀ to the (total) amount of fatty acid and/or diglyceride is not less than 1/19.

[42] The composition of the above-mentioned [38], which is a solid.

### EFFECT OF THE INVENTION

According to the present invention, a composition containing reduced coenzyme Q₁₀, which is stable against and suitable for oxidation especially without adding a plurality of ingredients, and a method for stabilization of reduced coenzyme Q₁₀ can be provided. Since the composition containing reduced coenzyme Q₁₀ of the present invention comprises, as constituent components thereof, reduced coenzyme Q₁₀ and general oil components (e.g., fat, oil, a mixture thereof etc.) conventionally used for various products such as pharmaceutical products and foods, it can be formulated into various kinds of preparations as it is or by mixing with other components as appropriate. In addition, the composition can be used by addition to common foods, and can be utilized in a very wide range of fields.

### BEST MODE FOR CARRYING OUT THE INVENTION

To suppress oxidation of reduced coenzyme Q₁₀ to oxidized coenzyme Q₁₀ by molecular oxygen, the present invention can afford (1) a liquid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/4, and (2) a solid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/9, whereby a composition containing reduced coenzyme Q₁₀, which is stable to oxidation, can be obtained.

In the present invention, reduced coenzyme Q₁₀ may be reduced coenzyme Q₁₀ alone or coenzyme Q₁₀ that is a mixture of oxidized coenzyme Q₁₀ and reduced coenzyme Q₁₀. When oxidized coenzyme Q₁₀ and reduced coenzyme Q₁₀ are used, a ratio of reduced coenzyme Q₁₀ to the total amount of coenzyme Q₁₀ (that is, the total amount of reduced coenzyme Q₁₀ and oxidized coenzyme Q₁₀) is not particularly limited, but is, for example, not less than 20 wt %, usually not less than 40 wt %, preferably not less than 60 wt %, more preferably not less than 80 wt %, particularly preferably not less than 90 wt %, and most preferably not less than 96 wt %. The upper limit is 100 wt %, usually not more that 99.9 wt %. Hereinafter, a case where “coenzyme Q₁₀” is only shown in the present specification refers the case of a mixture of oxidized coenzyme Q₁₀ and reduced coenzyme Q₁₀, and reduced coenzyme Q₁₀ alone.

In the present invention, the fat, oil or a mixture thereof to be used as an oil component is not particularly limited, and it may be natural fat or oil derived from animals and vegetables, or may be synthetic fat or oil or processed fat or oil. Specific examples of the vegetable fat or oil include coconut oil, palm oil, palm kernel oil, linseed oil, camellia oil, brown rice germ oil, rapeseed oil, rice oil, olive oil, peanuts oil, corn oil, wheat germ oil, soybean oil, perilla oil, cottonseed oil, sunflower oil, kapok oil, evening primrose oil, rhea butter, sal butter, cacao butter, sesame oil, safflower oil and the like, and examples of animal fat or oil include lard, milk fat, fish oil, beef fat and the like. Examples of the synthetic fat or oil include medium-chain triglyceride. In the present invention, these can also be used directly, or processed fat or oil obtained by processing by fractionation, hydrogenation, transesterification and the like can also be used. The fat or oil may be used alone, or in a mixture of two or more kinds. Examples of the medium-chain triglyceride include saturated or unsaturated fatty acid triglyceride having a carbon number of 6-12, preferably 10-12.

As the above-mentioned fat, oil or a mixture thereof, vegetable fat or oil, synthetic fat or oil and processed fat or oil are preferable from the aspects of easy handling, odor and the like. These are preferably selected in consideration of the price of fat and oil, stability of reduced coenzyme Q₁₀, solubility, property desired to be conferred to the composition and the like. From such aspects, for example, coconut oil, palm oil, palm kernel oil, camellia oil, brown rice germ oil, canola oil, rice oil, olive oil, peanuts oil, corn oil, wheat germ oil, soybean oil, perilla oil, cottonseed oil, sunflower oil, evening primrose oil, sesame oil, safflower oil, medium-chain triglyceride, and fat or oil obtained by processing them by partitioning, hydrogenation, transesterification and the like are preferable. More preferred are camellia oil, brown rice germ oil, canola oil, rice oil, olive oil, peanuts oil, corn oil, wheat germ oil, soybean oil, perilla oil, cottonseed oil, sunflower oil, evening primrose oil, sesame oil, safflower oil and medium-chain triglyceride, further preferred are brown rice germ oil, canola oil, rice oil, olive oil, corn oil, wheat germ oil, soybean oil, cottonseed oil, sunflower oil, safflower oil and medium-chain triglyceride, and particularly preferred are canola oil, rice oil, olive oil, soybean oil, safflower oil and medium-chain triglyceride.

In the present invention, while the fatty acid to be used as an oil component is not particularly limited, those having a carbon number of 8-22, more preferably 14-18, are preferably used. Specific examples thereof include caprylic acid, capric acid, lauric acid, myristic acid, palmitic acid, stearic acid, oleic acid, behenic acid, linoleic acid, linolenic acid and esters thereof, for example, methyl esters thereof, ethyl esters thereof and the like. Among these, myristic acid, palmitic acid, stearic acid, oleic acid, linoleic acid and linolenic acid are preferable, and palmitic acid, stearic acid and oleic acid are more preferably used. The above fatty acids may be used alone, or in a mixture of two or more kinds.

In the present invention, the wax to be used as an oil component is not particularly limited, and preferable examples thereof include beeswax, rice bran wax, carnauba wax, candelilla wax, sugar cane wax, shellac wax, jojoba wax and the like. Preferred are beeswax, rice bran wax, carnauba wax and shellac wax, and particularly preferred are beeswax and shellac wax. Among these, beeswax is preferable.

In the present invention, the surfactant to be used as an oil component is not particularly limited, and examples include partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid, polyglycerol ester of condensed ricinoleic acid and the like.

The above-mentioned partial glyceride of fatty acid is not particularly limited, and saturated or unsaturated fatty acid monoglycerides and diglycerides each having a carbon number of 6-22, preferably 8-22, can be used. For example, monoglycerol fatty acid ester such as monoglycerol monocaprylate, monoglycerol monocaprate, monoglycerol monolaurate, monoglycerol monomyristate, monoglycerol monopalmitate, monoglycerol monostearate, monoglycerol monooleate, monoglycerol monolinoleate, monoglycerol monolinolenate, monoglycerol dicaprylate, monoglycerol dicaprate, monoglycerol dilaurate, monoglycerol dimyristate, monoglycerol dipalmitate, monoglycerol distearate, monoglycerol dioleate, monoglycerol dilinoleate, monoglycerol dilinolenate, monoglycerol dibehenate, monoglycerol dierucate and the like, monoglycerol fatty acid-organic acid ester such as monoglycerol caprylate succinate, monoglycerol stearate citrate, monoglycerol stearate acetate, monoglycerol stearate succinate, monoglycerol stearate lactate, monoglycerol stearate diacetyltartrate, monoglycerol oleate citrate etc. and the like can be mentioned.

The above-mentioned propylene glycol ester of fatty acid is not particularly limited and, for example, propylene glycol ester of fatty acid having a carbon number of 6-18, preferably 6-12, such as propylene glycol laurate, propylene glycol stearate, propylene glycol oleate and the like can be mentioned.

The above-mentioned phospholipid is not particularly limited and, for example, lecithin (phosphatidylcholine) such as egg-yolk lecithin, purified soybean lecithin and the like and a hydrogenated compound thereof, lysolecithin, phosphatidylethanolamine, phosphatidylserine, phosphatidylinositol, phosphatidylinositolamine, phosphatidylglycerol, phosphatidic acid, dicetyl phosphoric acid, cardiolipin, sphingomyelin, ceramide phosphoryl ethanolamine and ceramide phosphoryl glycerol can be mentioned, and a mixture thereof can be further used.

The above-mentioned sucrose ester of fatty acid is not particularly limited and, for example, one or more hydroxyl groups of sucrose is (are) each ester bonded with fatty acid having a carbon number of 6-22 can be mentioned. For example, sucrose laurate, sucrose myristate, sucrose palmitate, sucrose stearate, sucrose oleate, sucrose behenate, sucrose erucate and the like can be mentioned.

The above-mentioned sorbitan ester of fatty acid is not particularly limited and, for example, one wherein one or more hydroxyl groups of sorbitan is (are) each ester bonded with fatty acid having a carbon number of 6-18 can be mentioned. For example, sorbitan monolaurate, sorbitan monopalmitate, sorbitan monostearate, sorbitan monooleate and the like can be mentioned.

The above-mentioned polyoxyethylene sorbitan fatty acid ester is not particularly limited and, for example, polyoxyethylene sorbitan monopalmitate, polyoxyethylene sorbitan monostearate, polyoxyethylene sorbitan monooleate, polyoxyethylene sorbitan tristearate, polyoxyethylene sorbitan trioleate and the like, wherein 6 mol-20 mol ethylene oxide chain is added, can be mentioned.

The above-mentioned polyglycerol ester of fatty acid is not particularly limited and, for example, polyglycerol-containing polyglycerol having a degree of polymerization of 2 to 10 as a main component, and polyglycerol wherein one or more of the hydroxyl groups are ester bonded with fatty acid having a carbon number of 6-22 can be mentioned. Specifically, for example, hexaglycerol monocaprylate, hexaglycerol dicaprylate, decaglycerol monocaprylate, triglycerol monolaurate, tetraglycerol monolaurate, pentaglycerol monolaurate, hexaglycerol monolaurate, decaglycerol monolaurate, triglycerol monomyristate, pentaglycerol monomyristate, pentaglycerol trimyristate, hexaglycerol monomyristate, decaglycerol monomyristate, diglycerol monooleate, triglycerol monooleate, tetraglycerol monooleate, pentaglycerol monooleate, hexaglycerol monooleate, decaglycerol monooleate, diglycerol monostearate, triglycerol monostearate, tetraglycerol monostearate, pentaglycerol monostearate, pentaglycerol tristearate, hexaglycerol monostearate, hexaglycerol tristearate, hexaglycerol distearate, decaglycerol monostearate, decaglycerol distearate, decaglycerol tristearate and the like can be mentioned.

The above-mentioned polyglycerol ester of condensed ricinoleic acid is not particularly limited, for example, one containing polyglycerol having an average degree of polymerization of 2-10, and one containing polyricinoleic acid having an average condensation degree (average number of condensed ricinoleic acid) of 2-4. For example, tetraglycerol ester of condensed ricinoleic acid, pentaglycerol ester of condensed ricinoleic acid, hexaglycerol ester of condensed ricinoleic acid and the like can be mentioned.

In the present invention, one kind selected from the above-mentioned surfactants can be used, and two or more kinds thereof can be used in combination.

The above-mentioned fat, oil and a mixture thereof, fatty acid, wax and a surfactant contained in the composition of the present invention are preferably acceptable as components to be added to pharmaceutical products, foods or cosmetics.

The composition of the present invention is a composition containing reduced coenzyme Q₁₀ and at least one kind selected from the aforementioned oil component consisting of fat, oil, a mixture thereof, fatty acid, wax and a surfactant. In the composition of the present invention, to stably maintain reduced coenzyme Q₁₀ against oxidation, the weight ratio of reduced coenzyme Q₁₀ to the oil component needs to be not less than 1/4 when the composition is a liquid and not less than 1/9 when the composition is a solid. When a mixture of two or more kinds from fat, oil, a mixture thereof, fatty acid, wax and a surfactant is used as an oil component, the weight ratio of reduced coenzyme Q₁₀ to the total amount thereof as an oil component naturally needs to be not less than the above-mentioned numerical values. That is, the composition of the present invention is a liquid composition wherein the weight ratio of the content of reduced coenzyme Q₁₀ in the composition to the total content of fat, oil, a mixture thereof, fatty acid, wax and a surfactant in the composition is not less than 1/4, or a solid composition, wherein the weight ratio of the content of reduced coenzyme Q₁₀ in the composition to the total content of fat, oil, a mixture thereof, fatty acid, wax and a surfactant in the composition is not less than 1/9. The simplest constitution of the composition of the present invention is, needless to say, coenzyme Q₁₀, which is reduced coenzyme Q₁₀ or a mixture of oxidized coenzyme Q₁₀ and reduced coenzyme Q₁₀ and an oil component (fat, oil, a mixture thereof, fatty acid, wax, surfactant or a mixture thereof) alone. Reduced coenzyme Q₁₀ needs to be contained at a weight ratio of not less than 1/4 when the composition is a liquid and not less than 1/9 when the composition is a solid, to the total content of at least one kind selected from oil components consisting of fat, oil, a mixture thereof, fatty acid, wax and a surfactant.

The “liquid composition” means that the melting point of the composition as a whole is lower than room temperature and not only that the composition is in a molten or solution state at room temperature, but also the composition shows flowability in handling at room temperature. The “room temperature” in this case refers to a temperature range for general handling or preservation and, for example, 20° C. In other words, the “liquid composition” in the present invention means, for example, that the melting point of the composition as a whole is less than 20° C. and the composition is in a molten or solution state at 20° C., and the composition shows flowability at 20° C. In addition, when the composition of the present invention is a liquid, the weight ratio of reduced coenzyme Q₁₀ to the above-mentioned oil component (fat, oil, a mixture thereof, fatty acid, wax, surfactant or a mixture thereof) in the liquid composition needs to be not less than 1/4, so as to stably maintain reduced coenzyme Q₁₀. When the weight ratio of reduced coenzyme Q₁₀ to the oil component is too high, the composition may become a slurry with high viscosity, making handling difficult. Therefore, a preferable weight ratio of reduced coenzyme Q₁₀ to the content of the above-mentioned oil component in the composition is appropriately determined based on the kind, mixing ratio and the like of the oil component to be used. It is preferably not less than 1/2, more preferably not less than 1, further preferably not less than 3/2, particularly preferably not less than 2, especially preferably not less than 3. It is generally not more than 9, preferably not more than 7, more preferably not more than 6, particularly not more than 4.

The solid state of the “solid composition” means that the melting point of the whole composition is not less than room temperature and the composition is in a solid state at room temperature, and the composition does not show smooth flowability in handling at room temperature and has a certain shape and volume at room temperature, and also includes a flexible state such as clay and butter. The “room temperature” in this case refers to, as mentioned above, a temperature range for general handling or preservation and, for example, 20° C. In other words, the “solid composition” in the present invention means, for example, that the melting point of the composition as a whole is not less than 20° C. and the composition is in a solid state at 20° C., and the composition does not show flowability and has a certain shape and volume at 20° C. When the composition of the present invention is a solid, it is desirably in a solid state at preferably not less than 30° C., more preferably not less than 40° C., in order to stably maintain the solid state. In addition, when the composition of the present invention is a solid, the weight ratio of reduced coenzyme Q₁₀ to the oil component (fat, oil, a mixture thereof, fatty acid, wax, surfactant or a mixture thereof) in the composition needs to be not less than 1/9, so as to stably maintain reduced coenzyme Q₁₀, to impart appropriate hardness to the composition to facilitate its handling depending on the kind of the oil component (e.g., fat, oil, a mixture thereof, fatty acid, wax, surfactant and the like), and to utilize reduced coenzyme Q₁₀ in the composition. When the weight ratio is less than 1/9, the oxidation of reduced coenzyme Q₁₀ in the composition cannot be sufficiently suppressed. On the other hand, when the weight ratio of reduced coenzyme Q₁₀ to the oil component is too high, the obtained composition may become too hard depending on the composition of oil components such as fat, oil, a mixture thereof, fatty acid, wax, surfactant and the like. Such composition may be difficult to divide or triturate, or releasability from the container may become poor.

When the composition is dissolved for use, moreover, the dissolution may take time. Therefore, a preferable weight ratio of reduced coenzyme Q₁₀ to the content of the above-mentioned oil component in the composition is appropriately determined based on the kind, mixing ratio and the like of the oil component to be used. It is preferably not less than 1/8, more preferably not less than 1/6, further preferably not less than 1/4, particularly preferably 1/2 or more, especially preferably 1 or more, particularly not less than 3, among others not less than 4. It is generally not more than 9, preferably not more than 7, more preferably not more than 6, particularly preferably not more than 5.

As mentioned above, when the composition is liquid in the present invention, the weight ratio of reduced coenzyme Q₁₀ to the content of the above-mentioned oil component in the composition is not less than 1/4. On the other hand, when the composition is a solid, the weight ratio of reduced coenzyme Q₁₀ to the content of the above-mentioned oil component in the composition is not less than 1/9. When the composition is a solid, reduced coenzyme Q₁₀ present in the composition is not easily oxidized as compared to a liquid, and therefore, the weight ratio of reduced coenzyme Q₁₀ varies between a liquid composition and a solid composition.

Depending on the object of use of the composition of the present invention, other components can be added. Examples of such components include excipient, disintegrant, lubricant, binder, anticoagulant, absorption promoter, solubilizing agent of active ingredient, antioxidant, dye and the like. Needless to say, addition of other active ingredients other than reduced coenzyme Q₁₀ is not prevented. Such other components are also preferably those acceptable as components to be added to pharmaceutical products, foods or cosmetics.

The above-mentioned excipient is not particularly limited. For example, sucrose, lactose, glucose, cornstarch, mannitol, crystalline cellulose, calcium phosphate, calcium sulfate and the like can be used as an excipient.

The above-mentioned disintegrant is not particularly limited. For example, starch, agar, calcium citrate, calcium carbonate, sodium hydrogen carbonate, dextrin, crystalline cellulose, carboxymethylcellulose, tragacanth and the like can be used as a disintegrant.

The above-mentioned lubricant is not particularly limited. For example, talc, magnesium stearate, polyethylene glycol, silica and the like can be used as a lubricant.

The above-mentioned binder is not particularly limited. For example, hydroxypropylmethylcellulose, tragacanth, gelatin, gum arabic, polyvinylpyrrolidone, polyvinyl alcohol, polyacrylic acid, polymethacrylic acid, sorbitol and the like can be used as a binder.

While the above-mentioned anticoagulant is not particularly limited, for example, talc, light anhydrous silicic acid, water-containing silicon dioxide and the like can be mentioned.

While the above-mentioned absorption promoter is not particularly limited, for example, higher alcohols and the like can be used.

While the solubilizing agent for the above-mentioned active ingredient is not particularly limited, for example, organic acids such as fumaric acid, succinic acid, malic acid and the like, and the like can be mentioned.

While the above-mentioned antioxidant is not particularly limited, for example, tocopherol, vitamin A, vitamin C, β-carotene, astaxanthin, citric acid, sodium bisulfite, sodium thiosulfate, Sodium pyrrosulfite, lipoic acid, dihydrolipoic acid, a derivative thereof and the like can be mentioned.

While the above-mentioned dye is not particularly limited, for example, titanium oxide, food colors, ferric oxide red dye, safflower dye, caramel dye, gardenia dye, tar pigment, chlorophyll and the like can be mentioned.

While the other active ingredients besides the above-mentioned reduced coenzyme Q₁₀ are not particularly limited, for example, amino acids such as leucine, valine, isoleucine and the like, vitamins such as vitamin B, vitamin C, vitamin A, vitamin E, vitamin D and the like and a derivative thereof, carotenoids such as β-carotene, astaxanthin, rutin, lycopene and the like, minerals such as magnesium, iron, selenium, manganese, zinc and the like, polyunsaturated fatty acids such as docosahexaenoic acid, eicosapentaenoic acid and the like, polyphenols, peptides such as glutathione and the like, organic acids, saccharides, proteins and the like can be mentioned. Among these, some can also be used as the above-mentioned antioxidant, and they are expected to show both functions of an antioxidant and an active ingredient. Polyunsaturated fatty acids such as docosahexaenoic acid, eicosapentaenoic acid and the like also have a function as one kind of the “fatty acid”, which is the essential component in the present invention. Needless to say, the above-mentioned active ingredients can be used in a mixture of two or more kinds thereof.

As the reduced coenzyme Q₁₀ contained in the composition of the present invention, reduced coenzyme Q₁₀ per se may be added. In the composition, it may be reduced coenzyme Q₁₀ obtained by reducing oxidized coenzyme Q₁₀ using a reducing agent such as sodium hydrosulfite (sodium dithionite), ascorbic acid and the like. Generally, since the ingredients of the composition can be simplified and easily prepared, reduced coenzyme Q₁₀ per se is preferably added. When the composition of the present invention is a liquid, reduced coenzyme Q₁₀ in the composition may be dissolved in an oil component or may be present as slurry.

As reduced coenzyme Q₁₀ to be contained in the composition of the present invention, when reduced coenzyme Q₁₀ obtained by reducing oxidized coenzyme Q₁₀ with a reducing agent in the composition as mentioned above is used, fatty acid or diglyceride is preferably selected as an oily component since the reduction reaction proceeds smoothly. That is, a composition comprising reduced coenzyme Q₁₀ and fatty acid and/or diglyceride, wherein the reduced coenzyme Q₁₀ is obtained by reducing oxidized coenzyme Q₁₀ in the composition (hereinafter sometimes to be abbreviated as composition (A)), is also another preferable embodiment of the present invention.

While the fatty acid to be used for the composition (A) is not particularly limited, those having a carbon number of 8-22, more preferably 14-18, are preferably used. Specific examples thereof include caprylic acid, capric acid, lauric acid, myristic acid, palmitic acid, stearic acid, oleic acid, behenic acid, linoleic acid, linolenic acid and esters thereof, for example, methyl esters thereof, ethyl esters thereof and the like. Among these, myristic acid, palmitic acid, stearic acid, oleic acid, linoleic acid and linolenic acid are preferable, and palmitic acid, stearic acid and oleic acid are more preferably used. The above fatty acids may be used alone, or in a mixture of two or more kinds.

While the diglyceride to be used for the composition (A) is not particularly limited, saturated or unsaturated fatty acid diglycerides each having a carbon number of 6-22, preferably 8-22, can be used. Among those, monoglycerol dicaprylate, monoglycerol dicaprate, monoglycerol dilaurate, monoglycerol dimyristate, monoglycerol dipalmitate, monoglycerol distearate, monoglycerol dioleate, monoglycerol dilinoleate, monoglycerol dilinolenate, monoglycerol dibehenate and monoglycerol dierucate are preferable. The above diglycerides may be used alone, or in a mixture of two or more kinds, or in combination with the above fatty acid.

In the composition (A), the weight ratio of the total amount of reduced coenzyme Q₁₀ and oxidized coenzyme Q₁₀ (i.e., the total amount of reduced coenzyme Q₁₀ obtained by reducing oxidized coenzyme Q₁₀ in the composition, oxidized coenzyme Q₁₀ to that remained without being reduced even after the reduction reaction and oxidized coenzyme Q₁₀ resulting from re-oxidation of the obtained reduced coenzyme Q₁₀ in the composition) to the (total) amount of fatty acid and/or diglyceride is not particularly limited. However, it is preferably high, preferably not less than 1/19, more preferably not less than 1/9, further preferably not less than 1/6, particularly preferably not less than 1/3, especially preferably 1/2 or more, among others 1 or more, most of all not less than 3, in view of oxidization stability of the reduced coenzyme Q₁₀ in the composition. The upper limit is generally not more than 9, preferably not more than 7, more preferably not more than 6, particularly not more than 5.

While the composition (A) may be liquid or solid, it is preferably solid in view of oxidization stability of the reduced coenzyme Q₁₀ in the composition. The solid state means that the melting point of the whole composition is not less than room temperature and the composition is in a solid state at room temperature, and the composition does not show smooth flowability in handling at room temperature and has a certain shape and volume at room temperature, and also includes a flexible state such as clay and butter. The “room temperature” in this case refers to, as mentioned above, a temperature range for general handling or preservation and, for example, 20° C.

The reducing agent to be used for reducing oxidized coenzyme Q₁₀ to reduced coenzyme Q₁₀ in a preparation of the composition (A) is not particularly limited as long as it has a reducing ability. Since the composition obtained after reduction can be directly used as a product without removing the reducing agent, those acceptable as an addition component to foods and pharmaceutical product preparations are preferable. For example, ascorbic acid esters such as ascorbic acid, ascorbyl palmitate and the like, citric acid, tocopherol, dihydrolipoic acid and the like are preferable, and ascorbic acid or ascorbyl palmitate is particularly preferable.

The composition (A) may further contain fat, oil, a mixture thereof, wax, surfactant other than diglyceride and other components. As the fat, oil, a mixture thereof, wax and other components, those exemplified above can be mentioned, and those exemplified above as preferable ones are also preferably used here. As the surfactant other than diglycerides, the above-mentioned examples of the “surfactant” except diglycerides can be mentioned, and the above-mentioned preferable examples of the “surfactant” except diglycerides are preferable.

As the items relating to composition (A) other than the fatty acid, diglyceride, weight ratio of the total amount of reduced coenzyme Q₁₀ and oxidized coenzyme Q₁₀ to the (total) amount of fatty acid and/or diglyceride, solid state, reducing agent and other components that can be further added in the above-mentioned composition (A), those exemplified in the present specification and preferable examples thereof can be used

To produce a liquid composition containing reduced coenzyme Q₁₀ of the present invention, since reduced coenzyme Q₁₀ is solid at room temperature, the above-mentioned oil component is preferably liquid at room temperature (e.g., one having a melting point of less than 20° C.). Even when the oil component is solid at room temperature, when a liquid composition is obtained by combining with an oil component liquid at room temperature, such oil component can also be used. The method for obtaining the liquid composition containing reduced coenzyme Q₁₀ of the present invention includes mixing reduced coenzyme Q₁₀ and the above-mentioned oil component with, where necessary, other additive components as appropriate. The method including uniformly mixing them with heating at, for example, not less than 30° C., preferably not less than 50° C., and cooling the mixture to a temperature desired for handling or preservation, for example, ambient temperature, is preferable.

On the other hand, when the composition of the present invention is a solid at room temperature, reduced coenzyme Q₁₀ in the composition may be completely compatible with the oil component, may be dispersed as fine particles in the solid oil component.

To produce a solid composition containing reduced coenzyme Q₁₀ of the present invention, an oil component solid at room temperature (melting point is not less than 20° C.) is used as the above-mentioned oil component. Even when an oil component liquid at room temperature is used as the above-mentioned oil component, a solid composition can be obtained by combining with other oil component solid at room temperature, or adjusting the amounts of the oil component and reduced coenzyme Q₁₀. The method for obtaining the solid composition containing reduced coenzyme Q₁₀ of the present invention includes mixing reduced coenzyme Q₁₀ and the above-mentioned oil component with, where necessary, other additive components as appropriate. In this case, they may be kneaded in a solid state, or preferably heated to not less than the melting points of reduced coenzyme Q₁₀ and/or oil component, uniformly mixed in a liquid state, and cooled to a solidification temperature (for example, room temperature) to give a solid.

In addition, the solid composition of the present invention is preferably a solid having a volume at a certain level or above, since the surface area per composition weight becomes small, oxidation of reduced coenzyme Q₁₀ in the composition can be suppressed and the composition can be maintained stably. From such aspects, the volume of a solid composition containing reduced coenzyme Q₁₀ of the present invention is preferably not less than 0.02 cm³, preferably not less than 0.06 cm³, particularly preferably not less than 0.1 cm³, and most preferably not less than 1 cm³. While a greater volume of the solid composition is preferable, it is generally not more than 1 m³, preferably not more than 0.1 m³, more preferably not more than 0.05 m³, further preferably not more than 0.01 m³ from the aspects of handling property.

To minimize the surface area per unit weight of the composition, the shape of the composition is important. Having the same volume, the surface area becomes smaller when the shape is closer to a mass, particularly a spherical shape, which is preferable for the composition. Conversely, as the shape becomes thinner and/or longer, the surface area increases, which is not preferable for the composition. Thus, assuming a complete sphere having the same volume as the volume of the composition, the shape of the composition is recommended to be a steric mass having a surface area 1-fold to 2-fold, more preferably 1-fold to 1.3-fold, of the surface area of the complete sphere. A preferable specific shape of the steric mass having a surface area within the aforementioned range is not particularly limited. Examples thereof include spherical shape, cube, cuboid, cylinder, polygonal column and the like since production and handling are easy.

Furthermore, when the composition containing reduced coenzyme Q₁₀ of the present invention is a solid, not only reduced coenzyme Q₁₀ in the composition can be stably maintained as compared to a liquid composition thereof, but also even when reduced coenzyme Q₁₀ near the surface of the composition is oxidized, the surface of the composition can be scraped to use the inside thereof. Therefore, it is preferable when use of a composition containing reduced coenzyme Q₁₀, which has not been oxidized much (ratio of oxidized coenzyme Q₁₀ contained is low) is desired.

The composition of the present invention can be preserved, irrespective of the state thereof, by filling in a container such as plastic container, metal container, glass container and the like. While the method of filling is not particularly limited, when the composition is in a solid state, it may also be dissolved by heating, filled as a liquid and solidified in a container. When the composition is in a liquid state, it may be directly (as liquid) filled in a container. These methods are preferable since they are easily performed and minimize gaseous phase in the container. On the other hand, when the composition is in a solid state, the composition solidified in another container or apparatus is cut into a desired size to give, for example, dice-like products, which can be preserved in each of the above-mentioned containers. Alternatively, a dice-like or granular composition separately solidified to have a desired size may be placed in the above-mentioned container for preservation. Naturally, the container is preferably tightly sealed, and the filling and tightly sealing are more preferably performed under an oxygen-blocked atmosphere as mentioned below.

Examples of the above-mentioned plastic container include bottle, bag, box, drum made of high density polyethylene, low density polyethylene, branched chain polyethylene, polyethylene terephthalate), polyvinyl alcohol, polypropylene, polyvinyl chloride, polyvinylidene chloride, nylon etc. and the like.

Examples of the above-mentioned metal container include bottle, can and drum made of iron, aluminum, nickel, cobalt, zinc, copper, titanium, tin, chrome etc. and the like. Naturally, containers made of an alloy if these metals, such as stainless, brass and the like, may also be used.

Examples of the above-mentioned glass container include jar, bottle and the like made of soft glass, hard glass and the like.

Each of these plastic containers, metal containers, glass containers and the like is sometimes used in a composite form with other materials. For example, a bag made of a laminate film of polyethylene and polyvinylidene chloride, a bag made of an aluminum laminate film wherein low density polyethylene and aluminum foil are laminated, an enamel container wherein iron is coated with glass and the like can be mentioned. Among these, a container made of an oxygen impermeable material or a material with low oxygen permeability is preferable for efficiently suppressing oxidation of reduced coenzyme Q. Examples of the container made of a preferable material include a bag made of a laminate film of polyethylene and polyvinyl alcohol, a bag made of an aluminum laminate film wherein polyethylene and aluminum are laminated, a bag made of an aluminum vapor deposition film wherein aluminum is vapor deposited on poly(ethylene terephthalate), a bag made of a silica vapor deposition film wherein silica is vapor deposited on poly(ethylene terephthalate), a metal can, a polyethylene bottle or drum, a poly(ethylene terephthalate) bottle or drum, a polypropylene bottle or drum, a glass bottle and the like. When a container made of a material having comparatively low air-tightness such as polyethylene and the like is used, it is desirable to employ multiple packaging using two or more of the aforementioned containers, or a combination of two or more kinds including a container made of an oxygen-impermeable material or a material with low oxygen permeability.

In addition, when the composition containing reduced coenzyme Q₁₀ of the present invention is in a solid state, it can be packaged with a film. Packaging with a film includes the following embodiments.

(i) Packaging embodiment wherein the composition is simply wrapped with a film and the content, is sealed tightly.

(ii) Packaging embodiment wherein the composition is wrapped with a film, and the content is sealed tightly by applying a seal and the like to a press joint of the film and the like. In this case, the film to be used is preferably an oxygen impermeable material or a material with low oxygen permeability, i.e., oxygen permeation suppressive film.

To maximally exhibit the effect of the present invention, the composition of the present invention is preferably prepared and/or preserved under oxygen-blocked atmosphere. In addition, the above-mentioned filling in a container and packaging with a film are also preferably performed under an oxygen-blocked atmosphere. The deoxidation atmosphere can be achieved by replacing with an inert gas, decompression, boiling or using these in combination. It is preferred that the replacement by an inert gas, that is, an inert gas atmosphere be at least used. Examples of the inert gas include nitrogen gas, helium gas, argon gas, hydrogen gas and carbon dioxide gas etc., with preference given to nitrogen gas. A deoxidant may also be used to achieve an oxygen-blocked atmosphere, and particularly, enclosure of a deoxidant is preferable for packaging or double packaging with the above-mentioned film.

The composition of the present invention can be directly used as a composition containing reduced coenzyme Q₁₀ for pharmaceutical products, foods and the like. In addition, it can be used as a starting material for various products by mixing with other components as appropriate or processing. When the composition of the present invention is used as a starting material for a product, it is preferable to handle a certain amount of the composition in a package form during the distribution process or storage before production, not only from the handling property but also from the oxidation stability of reduced coenzyme Q₁₀. Accordingly, the present invention also provides a composition containing reduced coenzyme Q₁₀ in the form of a package for starting materials, which is convenient for distribution and storage. While the package form here is not particularly limited, the composition of the present invention may be filled in containers such as plastic container, metal container, glass container and the like as mentioned above and, when the composition of the present invention is a solid, it may be packaged with a film and the like. The weight of the composition of the present invention in these forms is preferably not less than 1 kg, more preferably not less than 10 kg, further preferably not less than 50 kg, per one unit package. On the other hand, in view of the stability of reduced coenzyme Q₁₀, the upper limit is not particularly limited. However, from the aspects of handling property, not more than 10 t is preferable, and not more than 1 t is more preferable. The composition has a volume of about 1000 ml-1100 ml per 1 kg at room temperature (e.g., 20° C.) whether it is a liquid or a solid.

In the present invention, the composition in the above-mentioned packaged form or a part thereof can be used as it is or used after pulverization and, where necessary, dissolved in other preparation components, added with other preparation components, or emulsified or powderized using other preparation components, after which divided in small portions and filled in a soft capsule or hard capsule, or formulated as tablet or powder. In addition, the composition of the present invention can also be added as it is, or added after an operation similar to those mentioned above as necessary, to foods such as margarine, yogurt, confectionery and the like.

The composition containing reduced coenzyme Q₁₀ of the present invention can be directly formulated into preparations or products, or added to carriers for pharmaceutical products or foods, or carriers for cosmetics, and provided as pharmaceutical products or foods, or cosmetics.

In addition, the composition containing reduced coenzyme Q₁₀ of the present invention can also be added to the above-mentioned pharmaceutical products, foods, cosmetics, animal drugs, animal feeds and the like.

### Examples

The present invention is explained in more detail in the following by referring to Examples, which are not to be construed as limitative.

The purity of reduced coenzyme Q₁₀, and the weight ratio of reduced coenzyme Q₁₀ and oxidized coenzyme Q₁₀ in the following Examples and the like were determined by high performance liquid chromatography (HPLC) analysis under the following conditions.

### [HPLC Analysis Conditions]

column: SYMMETRY C18 (manufactured by Waters) 250 mm (length), 4.6 mm (inner diameter), mobile phase; C₂H₅OH:CH₃OH=4:3 (volume ratio), detection wavelength; 210 nm, flow rate; 1 ml/min, retention time of reduced coenzyme Q₁₀; 9.1 min, retention time of oxidized coenzyme Q₁₀; 13.3 min.

### Production Example 1

Oxidized coenzyme Q₁₀ (100 g, purity 99.4%) and L-ascorbic acid (60 g) were added to 1000 g of ethanol, and the mixture was stirred at 78° C. to perform a reduction reaction. After 30 hr, the mixture was cooled to 50° C., and 400 g of ethanol was added while maintaining the mixture at 50° C. The ethanol solution (containing 100 g of reduced coenzyme Q₁₀) was cooled to 2° C. at a cooling rate of 10° C./hr with stirring to give a white slurry. The obtained slurry was filtered under reduced pressure, and wet crystals were washed successively with cold ethanol, cold water and cold ethanol, each of which had been cooled to 2° C. Then, the wet crystals were dried under reduced pressure at 20-40° C. and 1-30 mmHg to give white dry crystals (95 g, yield 95 mol). All operations except reduced-pressure drying were performed under a nitrogen atmosphere. The weight ratio of reduced coenzyme Q₁₀/oxidized coenzyme Q₁₀ of the obtained crystals was 99.5/0.5, and the purity of reduced coenzyme Q₁₀ was 99.2%.

### Example 1 and Example 2

The reduced coenzyme Q₁₀ obtained in Production Example 1 was added to medium-chain triglyceride (Actor M2; manufactured by RIKEN VITAMIN CO., LTD.; fatty acid composition C₈:C₁₀=99:1) to concentrations of 20 wt % and 40 wt %, and the mixtures were is each mixed at 50° C. to give liquid compositions as Example 1 and Example 2, respectively. The reduced coenzyme Q₁₀ obtained in Production Example 1 was added to medium-chain triglyceride in the same manner to concentrations of 1 wt % and 10 wt %, and the mixtures were each mixed at 50° C. to give liquid compositions as Comparative Example 1 and Comparative Example 2, respectively. 3 g each of the Examples and Comparative Examples was placed in a 30 ml glass container, and preserved at 40° C. for 14 days in the air without capping. The weight ratios of the reduced coenzyme Q₁₀/oxidized coenzyme Q₁₀ after the preservation are shown in Table 1.

TABLE 1

| | | reduced | reduced coenzyme |
|---|---|---|---|
| | content (wt %) | coenzyme Q₁₀/oil | Q₁₀/oxidized |
| | of reduced | component | coenzyme Q₁₀ |
| sample | coenzyme Q₁₀ | (weight ratio) | (weight ratio) |
| Example 1 | 20 | 1/4 | 96.5/3.5 |
| Example 2 | 40 | 2/3 | 97.5/2.5 |
| Comparative | 1 | 1/99 | 69.5/30.5 |
| Example 1 | | | |
| Comparative | 10 | 1/9 | 90.5/9.5 |
| Example 2 | | | |

As is clear from Table 1, in Example 1 and Example 2 of the present invention, wherein the weight ratios of reduced coenzyme Q₁₀ to the oil component were 1/4 and 2/3, reduced coenzyme Q₁₀ after the preservation for 14 days decreased slightly, and oxidation of reduced coenzyme Q₁₀ was shown to have been suppressed well. In contrast, in Comparative Example 1 and Comparative Example 2, wherein the weight ratios of reduced coenzyme Q₁₀ to the oil component were 1/99 and 1/9, not less than 30 wt % and not less than 9 wt %, of reduced coenzyme Q₁₀ had been oxidized, respectively, after the preservation for 14 days.

### Example 3

The reduced coenzyme Q₁₀ obtained in Production Example 1 was added to the above-mentioned oil component consisting of medium-chain triglyceride/beeswax=1/1 (weight ratio) to 20 wt % at 70° C., and the mixture was completely dissolved and filled in a cylindrical container (diameter 1.5 cm). After filling, the composition was solidified by cooling, taken out from the container and cut into 1 cm-thick rounds as Example 3. The reduced coenzyme Q₁₀ obtained in Production Example 1 was added to the above-mentioned oil component consisting of medium-chain triglyceride/beeswax=1/1 (weight ratio) to 5 wt % at 70° C. The solid composition obtained in the same manner was used as Comparative Example 3. Each of Example 3 and Comparative Example 3 was preserved at 40° C. for 28 days in the air. The weight ratios of the reduced coenzyme Q₁₀/oxidized coenzyme Q_{N} after the preservation are shown in Table 2.

TABLE 2

| | | | reduced coenzyme |
|---|---|---|---|
| | content (wt %) | reduced coenzyme | Q₁₀/oxidized |
| | of reduced | Q₁₀/oil component | coenzyme Q₁₀ |
| sample | coenzyme Q₁₀ | (weight ratio) | (weight ratio) |
| Example 3 | 20 | 1/4 | 95.5/4.5 |
| Comparative | 5 | 1/19 | 91.5/8.5 |
| Example 3 | | | |

As clear from Table 2, in Example 3 of the present invention, wherein the weight ratios of reduced coenzyme Q₁₀ to the oil component was 1/4, only less than 5 wt % of reduced coenzyme Q₁₀ had been oxidized after preservation at 40° C. for 28 days. However, in Comparative Example 3, wherein the weight ratio of reduced coenzyme Q₁₀ to oil component was 1/19, not less than 8 wt % of reduced coenzyme Q₁₀ had been oxidized.

### Example 4

The reduced coenzyme Q₁₀ (80 g) obtained in Production Example 1 and stearic acid (20 g) were mixed, and the mixture was heated to 50° C. The obtained oil composition was filled in a 150 ml polyethylene bottle, and left standing at room temperature (20° C.) as it was, whereby Example 4, which is a solid composition consisting of reduced coenzyme Q₁₀ and stearic acid (weight ratio of reduced coenzyme Q₁₀ to oil component-4/1), was obtained. The above-mentioned operations were performed under a nitrogen atmosphere and the container was sealed.

### Example 5

The reduced coenzyme Q₁₀ (20 g) obtained in Production Example 1 and hydrogenated oil (80 g) were mixed, and the mixture was heated to 80° C. The obtained oil composition was filled in a 120 ml aluminum bottle, and left standing at room temperature (20° C.) as it was, whereby Example 5, which is a solid composition consisting of reduced coenzyme Q₁₀ and hydrogenated oil (weight ratio of reduced coenzyme Q₁₀ to oil component=1/4), was obtained. The above-mentioned operations were performed under a nitrogen atmosphere and the container was sealed.

### Example 6

The reduced coenzyme Q₁₀ (10 kg) obtained in Production Example 1, canola hydrogenated oil (10 kg) and ascorbyl palmitate (10 g) were mixed, and the mixture was heated to 80° C. The obtained oil composition was filled in a cardboard completely applied with a polyethylene film, and left standing to at room temperature (20° C.) as it was, whereby a solid composition consisting of reduced coenzyme Q₁₀, canola hydrogenated oil and ascorbyl palmitate was obtained. The opening at the press joint was closed to cover the composition by folding the film, so that the composition would not come into contact with the cardboard, namely, so that the composition would not come into contact with the air, and the cardboard was sealed with a tape to give Example 6 (weight ratio of reduced coenzyme Q₁₀ to oil component=1/1.001).

### Example 7

The reduced coenzyme Q₁₀ (100 g) obtained in Production Example 1 and canola hydrogenated oil (490 g) were mixed, and the mixture was heated to 80° C. The obtained oil composition was filled in a 600 ml polyethylene container, and left standing at room temperature (20° C.) as it was, whereby a solid consisting of reduced coenzyme Q₁₀ and canola hydrogenated oil was obtained. The solid was taken out from the container and cut into 1 cm square dice to give Example 7 (weight ratio of reduced coenzyme Q₁₀ to oil component=1/4.9).

### Comparative Example 4

The reduced coenzyme Q₁₀ (500 g) obtained in Production Example 1 was directly heated to 80° C. The obtained oil composition was filled in a 600 ml polyethylene container, which was the same as that in Example 7, and left standing at room temperature (20° C.) as it was, whereby a solid composition consisting of reduced coenzyme Q₁₀ alone was obtained as Comparative Example 4. In Comparative Example 4, the solid composition was taken out from the container, and cut into a dice. However, it was hard and consequently disintegrated to give an amorphous mass and powder.

### Example 8

The solid composition containing reduced coenzyme Q₁₀ of Example 3 was preserved at 40° C. for 15 days and 28 days in the air. When the weight ratio of reduced coenzyme Q₁₀/oxidized coenzyme Q₁₀ after the preservation was measured, the surface of the solid was removed in a thickness of about 1 mm, and the weight ratio of reduced coenzyme Q₁₀/oxidized coenzyme Q₁₀ inside the solid composition was measured. The ratio was 99.4/0.6 for the both and the inside of the solid composition of Example 3 was shown to have scarcely been oxidized.

### Example 9 and Example 10

The reduced coenzyme Q₁₀ obtained in Production Example 1 was added to the oil component consisting of medium-chain triglyceride (Actor M2; manufactured by RIKEN VITAMIN CO., LTD.; fatty acid composition C₈:C₁₀=99:1)/beeswax=1/1 (weight ratio) to concentrations of 10 wt % and 15 wt % at 70° C., and the mixtures were each completely dissolved and filled in a cylindrical container (diameter 1 cm). After filling, the composition was solidified by cooling, taken out from the container and cut into 1 cm-thick rounds to give solid compositions containing reduced coenzyme Q₁₀ as Example 9 and Example 10. The reduced coenzyme Q₁₀ obtained in Production Example 1 was added to the above-mentioned oil component consisting of medium-chain triglyceride/beeswax=1/1 (weight ratio) to 5 wt % at 70° C. The solid composition obtained in the same manner was used as Comparative Example 5. Each of Example 9, Example 10 and Comparative Example 5 was preserved at 40° C. for 7 days in the air. The weight ratios of the reduced coenzyme Q₁₀/oxidized coenzyme Q₁₀ in the composition were measured and are shown in Table 3.

TABLE 3

| | content | | reduced coenzyme |
|---|---|---|---|
| | (wt %) of | reduced coenzyme | Q₁₀/oxidized |
| | reduced | Q₁₀/oil component | coenzyme Q₁₀ |
| sample | coenzyme Q₁₀ | (weight ratio) | (weight ratio) |
| Example 9 | 15 | 1/5.7 | 97.2/2.8 |
| Example 10 | 10 | 1/9 | 96.9/3.1 |
| Comparative | 5 | 1/19 | 93.1/6.9 |
| Example 5 | | | |

In Table 3, Example 9 directed to a solid composition wherein the weight ratio of reduced coenzyme Q₁₀ to the oil component was 1/5.7, and Example 10 directed to a solid composition, wherein the weight ratio of reduced coenzyme Q₁₀ to the oil component was 1/9, contained reduced coenzyme Q₁₀ oxidized alongside in a proportion of less than 3%. However, in Comparative Example 5, wherein the weight ratio of reduced coenzyme Q₁₀ to the oil component was 1/19, around 7% of reduced coenzyme Q₁₀ had been oxidized.

### Comparative Example 6

The reduced coenzyme Q₁₀ obtained in Production Example 1 was added to medium-chain triglyceride (Actor M2; manufactured by RIKEN VITAMIN CO., LTD.; fatty acid composition C₈:C₁₀=99:1) to a concentration of 15 wt %, and the mixture was mixed at 50° C. to give a liquid composition as Comparative Example 6.3 g of Comparative Example 6 was placed in a 30 ml glass container, and preserved at 40° C. for 14 days in the air without capping. The weight ratio of reduced coenzyme Q₁₀/oxidized coenzyme Q₁₀ after the preservation is shown in Table 4.

TABLE 4

| | content | reduced coenzyme | reduced coenzyme |
|---|---|---|---|
| | (wt %) of | Q₁₀/oil | Q₁₀/oxidized |
| | reduced | component | coenzyme Q₁₀ |
| sample | coenzyme Q₁₀ | (weight ratio) | (weight ratio) |
| Comparative | 15 | 1/5.7 | 92.2/7.8 |
| Example 6 | | | |

In Table 4, not less than 7% of reduced coenzyme Q₁₀ had been oxidized in the liquid composition of Comparative Example 6, wherein the weight ratio of reduced coenzyme Q₁₀ to the oil component was 1/5.7.

### Example 11

Oxidized coenzyme Q₁₀ (2 g, manufactured by KANEKA CORPORATION, purity 99.4%) and ascorbyl palmitate (5.8 g) were added to oleic acid (30 g), and the mixture was stirred at 80° C. for 48 hr to perform a reduction reaction, whereby a composition containing reduced coenzyme Q₁₀ was obtained. The obtained composition was liquid at room temperature. The composition was left standing in the air at 25° C. for 3 days in a glass reaction container without capping. As a result, the weight ratio of reduced coenzyme Q₁₀/oxidized coenzyme Q₁₀ in the composition was 99.2/0.8.

### Example 12

Oxidized coenzyme Q₁₀ (5 g, manufactured by KANEKA CORPORATION, purity 99.4%) and ascorbyl palmitate (14 g) were added to stearic acid (30 g), and the mixture was stirred at 80° C. for 48 hr to perform a reduction reaction, whereby a composition containing reduced coenzyme Q₁₀ was obtained. The obtained composition was solidified when left standing at room temperature. The composition was left standing as it was in the air at 25° C. for 3 days. As a result, the weight ratio of reduced coenzyme Q₁₀/oxidized coenzyme Q₁₀ in the composition was 98.6/1.4.

### Example 13

Oxidized coenzyme Q₁₀ (2 g, manufactured by KANEKA CORPORATION, purity 99.4%) and ascorbyl palmitate (5.8 g) were added to diacylglycerol (30 g, DGO manufactured by Nikko Chemicals: monoglyceroldioleic acid ester), and the mixture was stirred at 80° C. for 48 hr to perform a reduction reaction, whereby a composition containing reduced coenzyme Q₁₀ was obtained. The obtained composition was liquid at room temperature. The composition was left standing in the air at 25° C. for 3 days in a glass reaction container without capping. As a result, the weight ratio of reduced coenzyme Q₁₀/oxidized coenzyme Q₁₀ in the composition was 98.8/1.2.

### Comparative Example 7

Oxidized coenzyme Q₁₀ (2 g, manufactured by KANEKA CORPORATION, purity 99.4%) and ascorbyl palmitate (5.8 g) were added to medium-chain triglyceride (30 g), and the mixture was stirred at 80° C. for 48 hr to perform a reduction reaction, whereby a composition containing reduced coenzyme Q₁₀ was obtained. The obtained composition was liquid at room temperature. The composition was left standing in the air at 25° C. for 3 days in a glass reaction container without capping. As a result, the weight ratio of reduced coenzyme Q₁₀/oxidized coenzyme Q₁₀ in the composition was 35.5/64.5.

### INDUSTRIAL APPLICABILITY

The present invention can provide a composition containing reduced coenzyme Q₁₀, which is superior in oxidation stability, and a method of stabilizing reduced coenzyme Q₁₀ against oxidation.

While some of the embodiments of the present invention have been described in detail in the above, however, those of ordinary skill in the art can make various modifications and changes to the particular embodiments shown without substantially departing from the teaching and advantages of the present invention. Such modifications and changes are encompassed in the spirit and scope of the present invention as set forth in the appended claims.

This application is based on a patent application No. 2007-216896 filed in Japan (filing date: Aug. 23, 2007), the contents of which are incorporated in full herein.

1. A liquid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/4.

2. The composition according to claim 1, wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower oil, kapok oil, evening primrose oil, Shea butter, sal butter, cacao butter, sesame oil, safflower oil, olive oil, lard, milk fat, fish oil, beef fat, medium-chain triglyceride, and fat, oil and a mixture of fat and oil obtained by processing them.

3. The composition according to claim 1, wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

4. The composition according to claim 1, wherein the wax is at least one kind selected from the group consisting of beeswax, rice bran wax, carnauba wax, candelilla wax, sugar cane wax, shellac wax and jojoba wax.

5. The composition according to claim 1, wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid and polyglycerol ester of condensed ricinoleic acid.

6. The composition according to claim 1, which is filled in a container.

7. The composition according to claim 6, wherein the weight thereof per one package unit is not less than 1 kg.

8. A solid composition comprising reduced coenzyme Q₁₀ and at least one kind of oil component selected from the group consisting of fat, oil, a mixture thereof, fatty acid, wax and a surfactant, wherein the weight ratio of the reduced coenzyme Q₁₀ to the oil component is not less than 1/9.

9. The composition according to claim 8, wherein the fat, oil or a mixture thereof is at least one kind selected from the group consisting of coconut oil, palm oil, palm kernel oil, flaxseed oil, camellia oil, brown rice germ oil, canola oil, rice oil, peanuts oil, corn oil, wheat germ oil, soybean oil, perilla oil, cottonseed oil, sunflower oil, kapok oil, evening primrose oil, shea butter, sal butter, cacao butter, sesame oil, safflower oil, olive oil, lard, milk fat, fish oil, beef fat, medium-chain triglyceride, and fat, oil and a mixture of fat and oil obtained by processing them.

10. The composition according to claim 8, wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

11. The composition according to claim 8, wherein the wax is at least one kind selected from the group consisting of beeswax, rice bran wax, carnauba wax, candelilla wax, sugar cane wax, shellac wax and jojoba wax.

12. The composition according to claim 8, wherein the surfactant is at least one kind selected from the group consisting of partial glyceride of fatty acid, propylene glycol ester of fatty acid, phospholipid, sucrose ester of fatty acid, sorbitan ester of fatty acid, polyoxyethylene sorbitan fatty acid ester, polyglycerol ester of fatty acid and polyglycerol ester of condensed ricinoleic acid.

13. The composition according to claim 8, having a volume of not less than 0.02 cm³.

14. The composition according to claim 8, which is packed with a film.

15. The composition according to claim 8, which is filled in a container.

16. The composition according to claim 15, wherein the weight thereof per one package unit is not less than 1 kg.

17. A composition comprising reduced coenzyme Q₁₀ and fatty acid and/or diglyceride, wherein the reduced coenzyme Q₁₀ is obtained by reducing oxidized coenzyme Q₁₀ in the composition.

18. The composition according to claim 17, wherein the fatty acid is at least one kind selected from the group consisting of caprylic acid, capric acid, lauric acid, myristic acid, palmitic acid, stearic acid, oleic acid, behenic acid, linoleic acid, linolenic acid and esters thereof.

19. The composition according to claim 17, wherein the diglyceride is at least one kind selected from the group consisting of monoglycerol dicaprylate, monoglycerol dicaprate, monoglycerol dilaurate, monoglycerol dimyristate, monoglycerol dipalmitate, monoglycerol distearate, monoglycerol dioleate, monoglycerol dilinoleate, monoglycerol dilinolenate, monoglycerol dibehenate and monoglycerol dierucate.

20. The composition according to claim 17, wherein the weight ratio of the total amount of reduced coenzyme Q₁₀ and oxidized coenzyme Q₁₀ to the (total) amount of fatty acid and/or diglyceride is not less than 1/19.

21. The composition according to claim 17, which is a solid.
